(19) United States
(12) Patent Application Publication
(10) Pub. No.: US 2006/0100580 A1
(43) Pub. Date: May 11, 2006

(54) Device for medical infusions

(76) Inventor: Jost Muller, 32545 Bad Oeynhausen (DE)

Correspondence Address: WHITE, REDWAY & BROWN LLP, 1217 KING STREET, ALEXANDRIA, VA, 22314, US

(21) Appl. No.: 10/904,983

(22) Filed: Dec. 8, 2004

(30) Foreign Application Priority Data
Oct. 30, 2004 (DE) 20 2004 016 797.2

(51) Int. Cl. A61M 1/00; F04B 17/00; F04B 35/04

(52) U.S. Cl. 604/152; 604/153; 417/413.2; 417/410.1

(57) ABSTRACT

In a device for medical infusions, with a supply container for the infusion liquid to be infused and also with a connecting stub for a connecting tube to the infusion needle, a pump is provided in the supply container for the liquid to be infused.

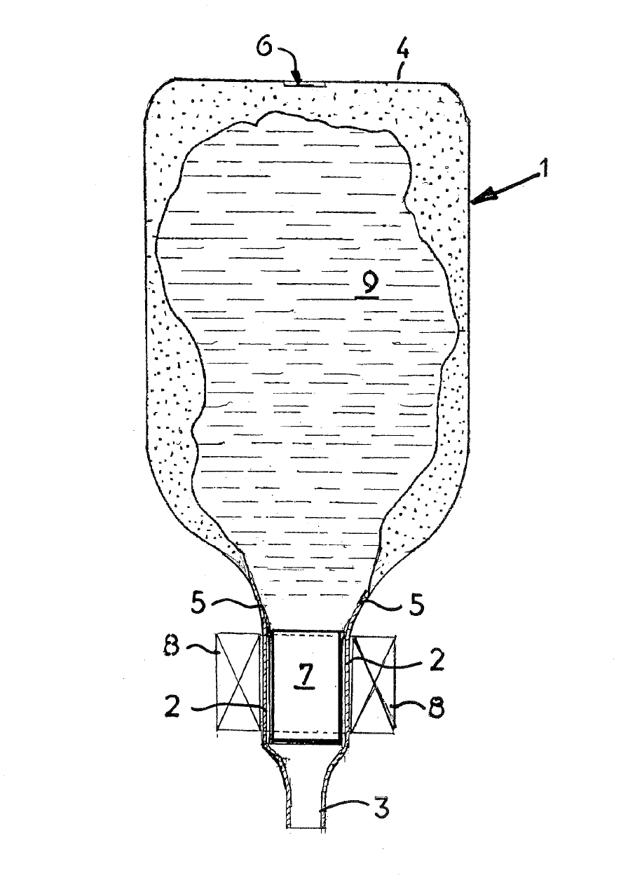

The invention relates to a device for medical infusions with a supply container for the liquid to be infused and also with a connecting stub for an infusion tube with needle.

Infusions are widely used in medical therapy, for example with cytostatic agents in oncology, with pain blockers in pain therapy, with hormones in reproductive therapy, or with insulin in diabetic therapy. These include infusions which have to be given very slowly over a long period or else metered. This is only possible in an unsatisfactory way with the known gravity infusions, such as described for example in DE 296 10 419, since the patient is not to be confined to bed over the long infusion times; rather, mobility is desired. To maintain the infusion, the supply container for the liquid to be infused is suspended on a rolling stand, with which the patient can move somewhat freely. For infusions during the transportation of recumbent patients, there can be problems with a gravity infusion because of the necessary suspension height of the supply container for the infusion liquid. Furthermore gravity infusion systems are personnel-intensive because of the required constant monitoring by the medical personnel in case of hindering of the patient supply.

Pump-supported infusion devices are also basically known. DE 199 281 33 describes a pump device for medical or nutrient-physiological liquids which is connected between a supply container for the infusion liquid and the infusion needle. Separable connections at the input and also the output of the pump device are then necessary.

From this there is derived the statement of object, to propose a device for medical infusions which is independent of gravity, which overcomes these disadvantages of the prior art and in particular reduces the number of necessary separable connections required to a minimum.

According to the invention, this statement of object is attained for a device according to the preamble of the independent claim by the features of its characterizing part; advantageous developments and preferred embodiments are described in the claims dependent thereon.

The placing of the pump in the supply container for the infusion liquid permits an infusion without the height difference necessary for gravity infusion between the supply container for the infusion liquid and the position of the infusion needle. Furthermore an insertion of the pump between the supply container for the infusion liquid and the infusion needle is omitted. The patient being infused is thereby more freely mobile; it is no longer necessary to bring along a stand for the supply container for the infusion liquid, whose releasable connection to the pump is likewise superfluous.

The material for such a pump arranged in the container can be adapted to the requirements; preferably plastics are used. A separate sterilization of the pump is dispensed with because of its placing in the supply container for the infusion liquid. The danger of transmission of infection due to repeated use of this pump is excluded from the beginning onward.

All pump systems which can be inserted in the liquid may be used as the pump. The use of rotating pumps is also possible, as is the use of piston pumps, displacement pumps, peristaltic pumps, and the like, the drive means provided on the pump being excited from outside.

For excitation, the effect of spring force, or hydraulic or pneumatic pressure may be used. If the neck of the supply container for the infusion liquid is formed as a flexible cylinder, it can be enclosed in a kind of milking machine stub which forwards the liquid to be infused to the infusion needle with pneumatic or hydraulic means.

In another embodiment, magnetic or electromagnetic alternating effects are used in order to forward the infusion liquid to the infusion needle. Accordingly the runner, formed as a rotor, of a rotation pump can for example be excited by means of a rotating electromagnetic field, or in the sense of a unipolar machine, with a magnetic field. A piston movement can be effected with alternating magnetic fields if the piston as formed as an armature in a coil and reciprocates the coil longitudinally by applying current to the coil.

Miniaturized plastic pumps are particularly advantageous; polycarbonate has been found to be particularly suitable. For excitation, the piezo effect of a ceramic plate excited by an alternating voltage is used. The arrangement within the supply container for the infusion liquid requires an energy supply without wires, which is possible in a simple manner with the necessary medium or high frequency voltages. The infusion speed can then be adjusted using the frequency and/or the amplitude of the exciting voltage.

Metering possibilities are given by such pumps. With rotating pumps, metering is effected by means of regulating the stroke or rpm. Piston pumps permit very accurate metering, their stroke being calibrated, and their stroke also being given at defined time intervals by corresponding control of the current acting on the coil. With piezo pumps, time intervals of excitation and/or frequency or amplitude of the exciting voltage likewise act to give the desired adjustment of the dose.

The nature of the invention will be explained in detail with reference to the accompanying FIGURE.

This FIGURE shows a device for pump-supported infusion of medical liquids.

The supply container 1 for the infusion liquid 9 contains the pump 7, which is arranged in the region of the outlet neck 2 of the supply container 1. The wall 5 of the supply container 1 can be of rigid or else flexible construction, where with a rigid wall 5 a ventilation 6 is provided in the region of the floor 4 through which air entry can compensate for the reduced pressure occurring during infusion due to the infusion liquid 9 being sucked out. The outlet neck 2 of the supply container 1 is adapted to the shape of the pump 7, which is shown as cylindrical in the FIGURE. Its outlet merges into the connecting stub 3 for the infusion tube (not shown). The pump 7 is arranged in the outlet neck 2, and is surrounded by the external device 8 effecting the excitation. For example, this produces a magnetic or electromagnetic field by means of a coil, to excite the runner of the pump 7 to a rotary motion, the piston of a pump to a reciprocating motion, or a piezo pump plate to a thickness change or to a bending vibration. With the pump 6, the infusion liquid 9 is drawn out of the supply container 1 corresponding to the setting, to be selected according to requirements, of the amount to be forwarded by the pump 7 out of the supply container, according to requirements, and is forced into the infusion tube to be connected to the connecting stub 3.

List of Features
01 Supply container for the infusion liquid
02 Outlet neck
03 Connecting stub for the infusion tube
04 Floor
05 Wall
06 Ventilation
07 Pump
08 Excitation means for drive
09 Infusion liquid

What is claimed is:

1. Device for medical infusions, with a supply container for the infusion liquid to be infused and also with a connecting stub for a connecting tube to the infusion needle, wherein a pump is provided in the supply container for the liquid to be infused.

2. Device according to claim 1, wherein a displacement pump, for example a piston pump, a peristaltic pump, a micro-pump with piezo pump member, or the like is provided as the pump, the excitation means of which operating the drive is arranged outside the supply container.

3. Device according to claim 2, wherein spring force or hydraulic or pneumatic pressure force is provided as the direct or indirect drive means for the pump.

4. Device according to claim 2, wherein magnetic or electromagnetic alternating forces are formed for producing the exciting means.

5. Device according to claim 1, wherein a rotating pump is provided as the pump, whose exciting means acting on the pump runner is arranged outside the supply container.

6. Device according to claim 5, wherein spring force or hydraulic or pneumatic pressure force is provided as the direct or indirect drive means for the pump.

7. Device according to claim 5, wherein magnetic or electromagnetic alternating forces are formed for producing the exciting means.

8. Device according to claim 5, wherein as the pump a rotary pump is provided, the runner of which, formed as a rotor, can be set in rotation by means of an electromagnetic rotary field applied from outside by the exciting means, or in the sense of a unipolar machine with a magnetic field, valves preventing backflows preferably being provided in the inflow region and/or in the outflow region of the pump.

9. Device according to claim 1, wherein a displacement pump, piston pump or the like is provided as the pump, the piston of which, formed as an armature, can be set in reciprocating motion by means of a magnetic alternating field produced by exciting means formed as a coil, valves preventing backflow preferably being provided in the inflow region and/or the outflow region of the pump.

10. Device according to claim 1, wherein a micro-pump with piezo pump member, acting as a displacement pump, is provided as the pump, the piezo member of which can be excited by means of an external alternating electromagnetic field, valves preventing backflow preferably being provided in the inflow region and/or the outflow region of the pump.

11. Device according to claim 10, wherein the piezo member of the pump is a thickness vibrator, the thickness vibrations of which displace the infusion liquid to be forwarded in the direction of the connecting stub for the infusion tube, valves preventing backflow preferably being provided in the inflow region and/or the outflow region of the pump.

12. Device according to claim 10, wherein the piezo member is a bending vibrator, the bending vibrations of which displace the infusion liquid to be forwarded in the direction of the connecting stub for the infusion tube, valves preventing backflow preferably being provided in the inflow region and/or the outflow region of the pump.

13. Device according to claim 1, wherein the pump is substantially produced from plastic, preferably polycarbonate.
